(19) United States
(12) Patent Application Publication
(10) Pub. No.: US 2013/0035399 A1
(43) Pub. Date: Feb. 7, 2013

(54) MARKED INORGANIC ADDITIVES

(71) Applicant: SACHTLEBEN CHEMIE GMBH, Duisburg (DE)

(72) Inventors: Ralf Eickschen, Moers (DE); Joerg Hocken, Meerbusch (DE); Juergen Kastner, Bochum (DE)

(73) Assignee: SACHTLEBEN CHEMIE GMBH, Duisburg (DE)

(21) Appl. No.: 13/624,025

(22) Filed: Sep. 21, 2012

Related U.S. Application Data
(63) Continuation of application No. 11/988,596, filed on Mar. 31, 2008, now abandoned.

(30) Foreign Application Priority Data
Jul. 13, 2005 (DE) 102005033208.0

(51) Int. Cl. C01G 23/047; C01G 9/08; A61K 47/02; A23L 1/03; C03C 14/00; C09D 1/00; C08K 3/22; C08K 3/30; C01F 11/46; A61K 8/19

(52) U.S. Cl. 514/769; 423/610; 423/554; 423/566.1; 501/32; 106/420; 106/438; 106/442; 106/446; 106/436; 106/466; 106/461; 524/413; 524/420; 524/423; 426/531

(57) ABSTRACT

The subject matter of the present invention is marked inorganic additives, a method for their production and also their use.

The subject matter of the present invention is marked inorganic additives, a method for their production and also their use.

In particular, the subject matter of the present invention is marked inorganic pigments and fillers, a method for their production and also their use.

In the case of foodstuffs, feedstuffs, drugs, yet also in the case of many industrial products, the need exists to have clear proof of origin. Thus, for example, for complaints purposes it is necessary to prove from which manufacturer a raw material has been supplied. By the same token, the suppliers want to have the possibility of protecting themselves, by means of clear proof of origin, from claims for damages falsely directed at them. In medicine, for example, special synthetic barium sulphates are used for the field of application of X-ray contrast media. In developing countries, from time to time barium sulphate products that are of inferior quality (instances of plagiarism) crop up for the application specified. When administering these products, it is impossible to preclude a threat to the health of the patients. It is therefore in the interest of the manufacturers of high-quality products to be able to prove the use of their own products beyond all doubt and in the smallest quantities in order to be able to protect themselves, if necessary, from unjustified rights of recourse.

It is known from the prior art that the substances supplied can be marked by adding to them substances that have an increased proportion of heavy, stable isotopes. In all chemical compounds, the chemical elements, out of which the corresponding compound is built up, are composed of various stable isotopes. These stable isotopes occur naturally and thus also in the substances that are to be marked in natural widths of distribution, so-called “average natural frequencies” (Table 1, Chiuz. 2005, 39, page 93) so that very high concentrations of the marked substances have to be added, in particular admixed, in order to effect a significant deviation.

TABLE 1
“Average natural frequencies” of selected isotopes

| | Isotope | | Name | |
|---|---|---|---|---|
| Element | Symbol | F [atom-%] | (Abbreviation) | Standard R |
| Hydrogen | ¹H | 99.9855 | Standard Mean | 0.00015576 |
| | ²H | 0.0145 | Ocean Water | |
| | | | (V-SMOW) | |
| Carbon | ¹²C | 98.892 | PEE Dee | 0.0011237 |
| | ¹³C | 1.1080 | Belemnite | |
| | | | (V-PDB) | |
| Nitrogen | ¹⁴N | 99.6337 | Air Nitrogen | 0.0036765 |
| | ¹⁵N | 0.3663 | (Air) | |
| Oxygen | ¹⁶O | 99.7587 | Standard Mean | 0.0020052 |
| | ¹⁷O | 0.0375 | Ocean Water | |
| | ¹⁸O | 0.2039 | (V-SMOW) | |
| Sulphur | ³²S | 95.0180 | Canyon Diablo | 0.0450045 |
| | ³³S | 0.7500 | Troilite | |
| | ³⁴S | 4.2150 | (CDT) | |
| | ³⁵S | 0.0200 | | |

The elements and their stable isotopes that are of significance for marking pigments or fillers, their relative average frequencies F (F=[isotope]/[Σisotope]), the name of the international isotope standard and its isotopic ratio R for the two most frequent stable isotopes (R=[isotope a]/[isotope b]) are specified in the table. In practice, however, there are slight local and temporal deviations from the frequencies specified for each element in the chemical compounds. That is why the precise isotopic concentrations of all the samples are specified as relative differences with respect to those international standards in δ-values in per thousands.

That means that in the first instance the δ-values are determined for the relevant isotopes of the various pigments and fillers (for example: isotopic ratio ¹⁸O/¹⁶O in comparison with the corresponding standard V-SMOW for BaSO₄ or isotopic ratio ³⁴S/³²S in comparison with the corresponding standard CDT for ZnS), this then being established as the natural isotopic ratio for this compound under the conditions applied (raw materials used, manufacturing conditions).

In the case of the marking in accordance with the prior art, account must be taken of the problems with regard to the fact that the proportion of the marking isotopes in the substance to be marked can be diminished by process-engineering steps, for example. Thus samples whose origin is to be identified may possibly have been diluted. This is the case in particular with feedstuffs and foodstuffs, for example fruit juices. When adding, for example, isotope-marked water, this can be withdrawn from the substance that is to be marked as a result of drying or the like. Even after dilution or drying, the concentration of stable isotope must always still lie significantly above or below the naturally occurring concentration so that it is possible to prove the origin. The consequence is that it is necessary to use correspondingly high concentrations of marked substance. However, this results in a high consumption of marked substance and, connected with this, high costs. Furthermore, in particular in the case of foods as a result of undesirably high concentrations of marked substance toxic side-effects can occur that are not acceptable. If dilution is arbitrarily high, the marking can possibly no longer be measured.

From the point of view of measuring techniques, this problem has been solved in part by the technical teaching of DE-A-102 00 802. However, this method only relates to a marking with deuterated water. Thus this method has the disadvantage that it can only be used if the water that is introduced at least in part remains permanently in the end product. This method cannot therefore be used for marking inorganic additives, in particular pigments and fillers. During their production and processing these are repeatedly dried or subjected to heat treatment, during which a temperature of 100° C. is often exceeded, for example when extruding polymer materials.

The object of the present invention is to overcome the disadvantages of the prior art.

In particular the object of the present invention is to provide an inorganic additive which is permanently marked by one or a plurality of heavy, stable isotopes and a method for its production. A further object of the invention is to provide an inorganic additive, whose content of the isotope used for marking or of the isotopes used for marking cannot be affected by heat treatment.

In accordance with the invention this problem is solved surprisingly by means of the features of the main claim. Advantageous developments are found in the subclaims.

So that when producing the end product it is not possible for the marking to be changed or lost, the marking must be incorporated in the chemical compound of the additive in a permanent or fixed manner. If, for example, an inorganic additive such as TiO₂, which can be used, for example, as a white pigment, is moistened before use just with O¹⁸-marked water, it is possible that when the end product is produced the marking will only be introduced into the end product in part or will not be introduced into it at all. This can always happen when using marked water if a thermal or water-removing step is included in the process of production of the additive or of the end product with use of the additive.

The marking with heavy stable isotopes must be selected so that the content in the end product can be proved beyond all doubt (for example by means of stable isotope mass spectrometry), even if only a few milligrams of the end product are available.

In this connection, the problem is solved in accordance with the invention by means of an inorganic additive which in comparison with the natural occurrence has an increased proportion (Δδ has positive values) or reduced proportion (Δδ has negative values) of heavy stable isotopes, for example ²H, ¹³C, ¹⁵N, ¹⁸O and/or ³⁴S. The inorganic additive is preferably a pigment or a filler. Examples of such an inorganic additive are TiO₂, ZnS or BaSO₄.

The marking is fixedly incorporated in the additive. Fixedly incorporated in this connection means that the respective Δδ has a constant value for a particular additive. In addition, a particular content of heavy isotopes, that is, a particular Δδ, can be set in a specific way.

In this case, Δδ denotes the difference in the δ-values of the compound enriched with heavy isotopes and the “natural” compound that is not enriched (for example δ³⁴S of ZnS marked with ³⁴S minus δ³⁴S of “natural” ZnS).

In accordance with the invention, the Δδ-values lie between 3 and 1000‰, preferably between 5 and 300‰, particularly preferably between 10 and 200‰, and/or between −3 and −100‰, particularly preferably between −5 and −20‰.

As a result of the increased or reduced proportion of heavy isotopes, for example ²H, ¹³C, ¹⁵N, ¹⁸O and/or ³⁴S, these substances can be clearly and permanently marked and detected. In the case of substances that are marked by two or more isotopes, the isotopes that are used for marking can all be enriched or all be depleted or one or more isotopes can be enriched, whilst the other isotope or the other isotopes is or are depleted.

The marking can be effected by means of various methods, for example:
The inorganic additive is permanently marked directly during production by the incorporation of a suitable heavy stable isotope in the chemical compound.
Inorganic additives, in particular pigments and fillers, are often provided with inorganic and/or organic aftertreatment and in particular are coated. As a result of these aftertreatments, the properties of the additives are changed in such a way that they are optimally adapted for the intended purpose (Jochen Winkler, Titandioxid, chapter 3.4, Hanover, Vicentz 2003; Elizabeth Reck, Farbe&Lack, 102nd volume 3/96, pages 40-48). This inorganic and/or organic aftertreatment can also be used for permanent marking with heavy stable isotopes.
Fillers and pigments, such as, for example, TiO₂, ZnS or BaSO₄ (inorganically aftertreated or not aftertreated), can be marked by specific contamination with thermostable, inorganic substances that are difficult to dissolve with a changed isotope content of ¹³C, ¹⁸O, ¹⁵N, and/or ³⁴S. Oxygen-containing inorganic Al—, Ba—, Si—, Ti—, Zr—Ca—, Mg—, Fe—, Zn— and/or B-compounds that are difficult to dissolve are suitable for these contaminations, for example. These inorganic compounds need not necessarily be of an oxidic nature, but can also contain, for example, anions, such as hydroxides, phosphates, carbonates, nitrates, sulphides or even sulphates.

Any combinations of the methods mentioned are also possible. By means of these methods the inorganic additive is permanently marked by heavy isotopes.

The inorganic additives that are marked by means of the methods described above can be applied to all fields of use for additives, for example as pigments and/or fillers, in turn for example in:
plastics/polymer materials (for example PET-bottles, film/foils, synthetic and chemical fibres);
b) paints and lacquers;
c) foodstuffs;
d) pharmaceutical agents;
e) cosmetic agents;
f) paper;
g) rubber;
h) glass;
i) feedstuffs.

The subject matter of the invention in detail is:
an inorganic additive that is marked by heavy, stable isotopes;
an inorganic additive that is marked by heavy, stable isotopes, with the marking being fixedly incorporated in the additive;
an inorganic additive that is permanently marked by heavy, stable isotopes;
an inorganic additive whose content of the isotope used for marking or of the isotopes used for marking cannot be affected by heat treatment;
an inorganic additive that is marked by heavy, stable isotopes, with the isotopes being selected from one or a plurality of ²H, ¹³C, ¹⁵N, ¹⁸O and ³⁴S;
an inorganic additive that is marked by heavy, stable isotopes, with the isotopes being enriched;
an inorganic additive that is marked by heavy, stable isotopes, with the isotopes being depleted;
an inorganic additive that is marked by heavy, stable isotopes, with the additive being marked as such;
an inorganic additive that is marked by heavy, stable isotopes, with the coating of the additive being marked;
an inorganic additive that is marked by heavy, stable isotopes that are contained in specific contaminations of the additive;
an inorganic additive that is marked by heavy, stable isotopes, with the Δδ-values lying between 3 and 1000‰, preferably between 5 and 300‰, particularly preferably between 10 and 200‰, and/or between −3 and −100‰, particularly preferably between −5 and −20‰;
an inorganic additive that is marked by heavy, stable isotopes, with the additive being a pigment and/or a filler;
an inorganic additive that is marked by heavy, stable isotopes, with the additive being TiO₂, ZnS or BaSO₄;
a method for producing a marked inorganic additive;
a method for producing a marked inorganic additive, with the marking being fixedly incorporated in the additive;
a method for producing a marked inorganic additive, with the marking being effected by one or a plurality of heavy, stable isotopes and/or their compounds;
a method for producing a marked inorganic additive, with the isotopes being selected from one or a plurality of ²H, ¹³C, ¹⁵N, ¹⁸O and/or ³⁴S;
a method for producing marked inorganic additives, with the isotopes being enriched;
a method for producing a marked inorganic additive, with the isotopes being depleted;
a method for producing a marked inorganic additive, with the additive being marked directly during production by the incorporation of a heavy, stable isotope in the chemical compound of the additive;
a method for producing a marked inorganic additive, with the additive being marked during inorganic and/or organic aftertreatment with heavy, stable isotopes;
a method for producing a marked inorganic additive, with the additive being marked by specific contamination with thermostable inorganic substances that are difficult to dissolve;
a method for producing a marked inorganic additive, with the additive being a pigment and/or a filler;
a method for producing a marked inorganic additive, with the additive being TiO₂, ZnS or BaSO₄;
a method for producing a marked inorganic additive, with the ¹⁸O-content in TiO₂ or BaSO₄ being increased;
a method for producing a marked inorganic additive, with the ³⁴S-content in ZnS or BaSO₄ being increased;
a method for producing a marked inorganic additive, with the ¹⁸O-content being increased in the inorganic and/or organic aftertreatment of TiO₂, ZnS or BaSO₄ by ¹⁸O-marked oxides or hydroxides of aluminium, silicon, zircon, manganese and/or titanium;
a method for producing a marked inorganic additive, with the ²H—, ¹³C—, ¹⁵N—, ¹⁸O— and/or ³⁴S-content being increased or reduced in the organic aftertreatment by means of surface treatment with polyalcohols, amino compounds and/or silicone oils with a changed isotope content of ²H, ¹³C, ¹⁸O, ¹⁵N and/or ³⁴S;
a method for producing a marked inorganic additive, with the ²H—, ¹³C—, ¹⁸O, ¹⁵N— and/or ³⁴S-content being increased or reduced by specific contamination with thermostable inorganic substances that are difficult to dissolve with a changed isotope content of ²H, ¹³C, ¹⁸O, ¹⁵N and/or ³⁴S, in particular with oxygen-containing inorganic Al—, Si—, Ti—, Zr—, Ca—, Mg—, Fe—, Zn— and/or B— compounds;
the use of the additive in accordance with the invention as a filler and/or pigment;
the use of the additive in accordance with the invention in the production and processing of plastics/polymer materials (PET-bottles, film/foils, synthetic and chemical fibres), paints and lacquers, foodstuffs; pharmaceutical agents, cosmetic agents, paper; rubber, glass, feedstuffs, preferably as a pigment and/or filler.

The invention is explained in greater detail by means of the following examples without being limited thereto:

### EXAMPLE 1

### Production of Marked Inorganic Additives

a. Increase in the ¹⁸O-content in TiO₂ or BaSO₄:
i. Production of TiO₂ by hydrolysis of titanium-containing compounds, such as titanium tetrachloride with ¹⁸O-marked water; the conversion results in a TiO₂₋product with a δ(¹⁸O)-value of +50‰; an unmarked TiO₂-product, on the other hand, has a δ(¹⁸O)-value of −2.7‰; the Δδ-value therefore amounts to +52.7‰;
ii. Production of TiO₂ by hydrolysis of titanium-containing compounds, such as titanyl-sulphate-solution with ¹⁸O-marked water;
iii. Production of BaSO₄ by precipitation from aqueous barium chloride solution with ¹⁸O-marked sulphuric acid; the conversion results in a BaSO₄-product with a δ(¹⁸O)-value of +33‰; an unmarked BaSO₄-product, on the other hand, has a δ(¹⁸O)-value of −3‰; the Δδ-value therefore amounts to +36‰.
b. Increase in the ³⁴S-content in ZnS or BaSO₄:
i. Production of ZnS by reaction of ZnO with ³⁴S-marked sulphuric acid to form zinc sulphate and subsequent conversion with Na₂S to form ZnS;
ii. Production of BaSO₄ by precipitation from aqueous barium chloride solution with ³⁴S-marked sulphuric acid.

### EXAMPLE 2

### Production of Inorganic Additives Marked in the Coating

a. Increase in the ¹⁸O-content in the inorganic and/or organic aftertreatment of TiO₂, ZnS or BaSO₄. During the inorganic aftertreatment, colourless inorganic compounds that are difficult to dissolve are precipitated onto the individual pigment particles. The oxides or hydroxides of aluminium, silicon, zircon, manganese and/or titanium are used, for example, for the surface treatment. These precipitation reactions can be carried out with the use of ¹⁸O-marked raw materials.
b. Increase in the ²H, ¹³C, ¹⁵N and/or ¹⁸O-content in the organic aftertreatment of TiO₂, ZnS or BaSO₄. Compounds, such as polyalcohols, amino compounds or even silicone oils are used, for example, for the surface treatment of fillers and pigments. These compounds also reach the end product with the additive and can thus be used for the marking.

### EXAMPLE 3

### Production of Marked Pigments or Fillers by Means of Specific Contamination

a. Contamination of TiO₂ with ¹⁸O-marked ZrO₂;
b. Contamination of ZnS with ¹³C-marked CaCO₃;
c. Contamination of BaSO₄ with ¹⁵N and/or ¹⁸O-marked MgNH₄PO₄;
d. Contamination of TiO₂ with ¹⁸O-marked BaSO₄; the addition of BaSO₄ ¹⁸O-marked with one % by weight to form a TiO₂ pigment gives rise to a δ(¹⁸O)-value of +162‰ in the contaminated end product; the addition of one % by weight unmarked BaSO₄ to form a TiO₂ pigment, on the other hand, gives rise to a δ(¹⁸O)-value of −3‰ in the contaminated end product; the Δδ-value therefore amounts to +165‰.

Any combinations of Examples 1 to 3 are also possible.

1-21. (canceled)

22. An inorganic additive having a marking of at least one heavy stable isotope, wherein the marking is fixedly incorporated in the additive.

23. An inorganic additive according to claim 22 that is at least one of a filler or a pigment.

24. An inorganic additive according to claim 22, wherein the inorganic additive comprises TiO₂, ZnS or BaSO₄.

25. An inorganic additive according to claim 22, wherein the at least one isotope is one or more of ²H, ¹³C, ¹⁵N, ¹⁸O or ³⁴S.

26. An inorganic additive according to claim 22, wherein the at least one isotope is enriched.

27. An inorganic additive according to claim 22, wherein the at least one isotope is depleted.

28. An inorganic additive according to claim 22, wherein the additive is marked as such.

29. An inorganic additive according to claim 22, wherein a coating of the additive is marked.

30. An inorganic additive according to claim 22 marked by specific contaminations.

31. An inorganic additive according to claim 22, wherein the Δδ-values lie between 3 and 1000%.

32. A method for the production of an inorganic additive according to claim 22, wherein the marking is fixedly incorporated in the chemical compound of the additive.

33. A method according to claim 32, wherein the inorganic additive is permanently marked directly during production by the incorporation of a suitable heavy stable isotope in the chemical compound.

34. A method according to claim 3, wherein a inorganic additive is permanently marked during an inorganic or organic aftertreatment with the heavy stable isotope.

35. A method according to claim 32, wherein the inorganic additives are permanently marked by specific contamination with thermostable inorganic substances that are difficult to dissolve.

36. A method according to claim 32, wherein the ¹⁸O-content in TiO₂ or BaSO₄ is increased.

37. A method according to claim 32, wherein the ³⁴S-content an ZnS or BaSO₄ is increased.

38. A method according to claim 32, wherein the ¹⁸O-content is increased in the inorganic or organic aftertreatment of TiO₂, ZnS or BaSO₄ by an ¹⁸O-marked oxide or hydroxide of at least one of aluminum, silicon, zircon, manganese or titanium.

39. A method according to claim 32, wherein the ²H—, ¹³C—, ¹⁵N—, ¹⁸O— or ³⁴S-content is increased in organic aftertreatment by surface treatment with a polyalcohol, amino compounds or silicone oils with a changed isotope content of ¹³C, ¹⁸O, ¹⁵N or ³⁴S.

40. A method according to claim 32, wherein the ²H—, ¹³C—, ¹⁸O—, ¹⁵N— or ³⁴S-content is increased by specific contamination with a thermostable inorganic substance that is difficult to dissolve with a changed isotope content of ²H, ¹³C, ¹⁸O, ¹⁵N or ³⁴S.

41. A composition comprising:
the inorganic additive according to claim 22; and
a plastic, a polymer material, a paint, a lacquer, a foodstuff; a pharmaceutical agent, a cosmetic agent, paper; rubber, glass or a feedstuff.
